(19) United States
(12) Patent Application Publication
(10) Pub. No.: US 2016/0345877 A1
(43) Pub. Date: Dec. 1, 2016

(54) BIOLOGICAL INFORMATION DETECTOR, BIOLOGICAL INFORMATION MEASUREMENT SYSTEM AND HYPOGLYCEMIA SYMPTOM OCCURRENCE TIMING STORING METHOD

(71) Applicants: Seiko Epson Corporation, Tokyo (JP); Educational Foundation Kyorin Gakuen, Tokyo (JP)

(72) Inventors: Jun Takeuchi, Matsumoto-shi (JP); Junichi Otsuka, Chikuma-shi (JP); Yoshikazu Sumitani, Mitaka-shi (JP); Hitoshi Ishida, Mitaka-shi (JP)

(21) Appl. No.: 15/162,708

(22) Filed: May 24, 2016

(30) Foreign Application Priority Data
May 25, 2015 (JP) 2015-105416

(51) Int. Cl. A61B 5/145; A61B 5/11; A61B 5/00

(52) U.S. Cl. CPC A61B 5/14532; A61B 5/681; A61B 5/7475; A61B 5/7425; A61B 5/11; A61B 5/743; A61B 5/486; A61B 2560/0475; A61B 2562/0219

(57) ABSTRACT

A biological information detector includes a biological information detector that detects biological information of a user, a storage that stores the biological information, a timepiece that indicates a current date-time, an input unit that receives an input operation, in which when a predetermined input operation is performed on the input unit, the time indicated by the timepiece is stored as an occurrence time of a hypoglycemia symptom.

The entire disclosure of Japanese Patent Application No. 2015-105416, filed May 25, 2015 is expressly incorporated by reference herein.

### BACKGROUND

1. Technical Field

The present invention relates to a biological information detector, a biological information measurement system and a hypoglycemia symptom occurrence timing storing method.

2. Related Art

A self blood-sugar measurement unit for measuring a blood-sugar level of a diabetic patient has been typically known (see, for instance, Patent Literature 1: JP-A-2003-302406).

The self blood-sugar measurement unit disclosed in Patent Literature 1 includes a detection unit for measuring a blood-sugar level of collected blood, a storage for digitalizing and storing measurement data of the detection unit, a means for sending the digitalized measurement data to an information terminal, and a means for sending the digitalized measurement data sent to the information terminal to a blood-sugar level management device. With the above arrangement, the digitalized measurement data of the blood-sugar level can be easily stored and managed.

It should be noted that, when a hypoglycemia symptom occurs to a user, it is demanded to analyze biological information such as the stored blood-sugar level to find the cause of the hypoglycemia symptom.

However, though the self blood-sugar measurement unit disclosed in Patent Literature 1 can store and manage the measurement data of the collected blood-sugar level, the self blood-sugar measurement unit is not necessarily carried by a user at the time of the occurrence of the hypoglycemia, so that it is difficult to know the time when the user feels the hypoglycemia symptom based on the measurement data. In such a circumstance, it is difficult to extract the time when the hypoglycemia symptom occurred from the measurement data.

### SUMMARY

The invention aims to solve at least a part of the above problem(s). An object of the invention is to provide a biological information detector, a biological information measurement system and a hypoglycemia symptom occurrence timing storing method that allow a user to record the occurrence time of the hypoglycemia symptom.

A biological information detector according to a first aspect of the invention includes: a biological information detector configured to detect biological information of a user; a storage configured to store the biological information; a timepiece configured to indicate a current date-time; and an input unit configured to receive an input operation, in which when a predetermined input operation is performed on the input unit, the time indicated by the timepiece is stored as an occurrence time of a hypoglycemia symptom.

According to the first aspect of the invention, since the time indicated by the timepiece can be stored in the storage as the occurrence time of the hypoglycemia symptom when the predetermined input operation is performed by the user. Accordingly, it is only necessary for the user to perform the predetermined operation in order to record the occurrence time of the hypoglycemia symptom. Thus, the cause of the hypoglycemia symptom can be easily studied based on the recorded time and the biological information at the recorded time.

In the first aspect, it is preferable that the predetermined input operation is a combination of a first operation and a second operation that are mutually different.

According to the above arrangement of the first aspect, since the time is not recorded unless the combination of different first operation and second operation is performed by the user, erroneous recordation of the time as the occurrence time of the hypoglycemia symptom can be restrained.

In the first aspect, it is preferable that the input unit includes a tap operation detection unit configured to detect a tap operation of the user and an operation unit comprising a button exposed to an outside, the first operation is the tap operation, and the second operation is an input operation on the button.

According to the above arrangement of the first aspect, since the time is recorded as the occurrence time of the hypoglycemia symptom when the tap operation and the input operation on the button are performed by the user, the erroneous recordation of the above time can be reliably restrained.

In the first aspect, it is preferable that a display configured to display a setting screen allowing a transition to an occurrence timing recordation screen configured to record the occurrence time of the hypoglycemia symptom when the first operation is performed is provided, in which the biological information detector is configured to store the time indicated by the timepiece as the occurrence time of the hypoglycemia symptom when the second operation is performed on the displayed setting screen.

According to the above arrangement of the first aspect, since the setting screen for transition to the occurrence timing recordation screen for recording the above time is displayed after the first operation is performed, the user can easily recognize that the above time can be recorded by performing the second operation on the displayed setting screen. Accordingly, the recordation operation is facilitated.

In the first aspect, it is preferable that a display configured to display a setting screen allowing a transition to an occurrence timing recordation screen configured to record the occurrence time of the hypoglycemia symptom when the first operation is performed is provided, in which the biological information detector is configured to store the time indicated by the timepiece as the occurrence time of the hypoglycemia symptom when the second operation is performed on the displayed setting screen.

In the first aspect, it is preferable that the display is configured to display a setting screen configured to display a plurality of items when the first operation is performed, one of the plurality of items being configured for the transition to the occurrence timing recordation screen and being displayed preferentially to the rest of the plurality of items.

According to the above arrangement of the first aspect, since the item for the transition to the occurrence timing recordation screen is displayed preferentially to the rest of the plurality of items on the setting screen, the user can record the above time by performing the second operation on the displayed setting screen. Accordingly, the operation for recording the above time can be rapidly performed.

In the first aspect, it is preferable that a body movement information detection unit configured to detect body movement information of the user and a storage configured to store the biological information and the body movement information are provided.

According to the first aspect, the study on the cause of the hypoglycemia symptom can be facilitated using not only the biological information detected by the biological information detection unit but also the body movement information detected by the body movement information detection unit.

A biological information measurement system according to a second aspect of the invention includes: the biological information detector according to the above first aspect; and an information processor configured to communicate with the biological information detector, in which the biological information detector comprises a time information sending unit configured to send time information indicating a time stored in the storage as the occurrence time of the hypoglycemia symptom. The information processor includes: an information acquisition unit configured to acquire information sent from the biological information detector; a processor-side display configured to display an input screen, on which a time of at least one of a meal, an exercise and medicine-dose of the user is capable of being inputted; and a time storage configured to store the time indicated by the time information acquired by the information acquisition unit and the inputted time.

According to the above second aspect of the invention, the same advantage(s) as that of the biological information detector according to the first aspect can be achieved. Further, since the information processor records the time information indicating the above time and time of at least one of the user's meal, exercise and medicine-dose inputted by the user, a more detailed study on the cause of the hypoglycemia symptom can be made based on the recorded time of the occurrence of the hypoglycemia symptom and the time of the at least one of meal, exercise and medicine-dose.

In the second aspect, it is preferable that a timing display screen generation unit configured to generate a timing display screen comprising a time table showing hours in a predetermined time period arranged in a chronological order, a hypoglycemia occurrence marker disposed in the time table correspondingly to the time indicated by the time information, and an active time marker disposed in the time table correspondingly to the time are provided, in which the processor-side display is configured to display the timing display screen.

According to the above arrangement of the second aspect, since the timing display screen including the time table showing the time arranged in a chronological order within the predetermined time period, and the hypoglycemia occurrence marker and the active time marker disposed in the time table is displayed, the occurrence time of the hypoglycemia symptom, the mealtime, exercise time and medicine-dose time can be more easily recognized by the user. Accordingly, the relationship between the time of the meal, exercise and medicine-dose and the time at which hypoglycemia occurred can be more easily understood.

In the second aspect, it is preferable that the time information includes a date to which the occurrence time of the hypoglycemia symptom belongs, the information processor includes a calendar display screen generation unit configured to generate a calendar display screen comprising a calendar showing date in a predetermined time period arranged in a chronological order and a hypoglycemia occurrence marker disposed in the calendar correspondingly at least to the date comprised in the time information, and the processor-side display is configured to display the calendar display screen.

According to the above arrangement of the second aspect, since the calendar display screen including the calendar in which the hypoglycemia occurrence marker is disposed in accordance with the date included in the date-time information on a calendar having the date fields within a predetermined time period arranged in a chronological order is displayed, the date on which the hypoglycemia symptom occurred can be easily recognized by the user.

A timing storing method according to a third aspect of the invention is performed using a biological information detector configured to measure biological information, the method including: storing a current time as an occurrence time of a hypoglycemia symptom when a predetermined input operation is performed.

According to the third aspect of the invention, the same advantages as those of the biological information detector according to the first aspect and the biological information measurement system according to the second aspect can be achieved. Further, the current time can be recorded as the occurrence time of the hypoglycemia symptom only by performing the predetermined input operation. Accordingly, the user can record the occurrence time of the hypoglycemia symptom without troublesome work (e.g. taking a note).

A biological information detector according to a fourth aspect of the invention includes: a biological information detection unit configured to detect biological information of a user; a body movement information detection unit configured to detect the body movement information and operation information of the user; a timepiece configured to indicate date and time; and a controller configured to acquire the biological information and the body movement information, in which the controller is configured to judge a condition of the user based on the biological information and the body movement information and, when it is detected that a predetermined operation is performed based on the operation information from the body movement information detection unit, judge the time indicated by the timepiece as the occurrence time of the hypoglycemia symptom.

In the fourth aspect, it is preferable that the status of the user comprises at least one of an attachment condition of the biological information detector, a type of an exercise performed by the user and an intensity of the exercise performed by the user.

In the fourth aspect, it is preferable that a storage configured to store the biological information, the body movement information and the occurrence time of the hypoglycemia symptom are provided.

In the fourth aspect, it is preferable that a communication unit configured to send the biological information, the body movement information and the occurrence time of the hypoglycemia symptom to the information processor is provided.

In the fourth aspect, it is preferable that the predetermined operation is a tap operation.

In the fourth aspect, it is preferable that the body movement information detection unit is an acceleration sensor, and the controller detects the tap operation based on an output signal from the acceleration sensor.

A biological information presentation system according to a fifth aspect of the invention includes: a biological information detector; and an information processing terminal, in which the biological information detection unit includes: a biological information detection unit configured to detect biological information of a user; a body movement information detection unit configured to detect body movement information of the user; a timepiece configured to indicate date and time; a control unit configured to acquire the biological information and the body movement information of the user, to judge a type and a time of an exercise performed by the user based on the biological information and the body movement information and to judge the time indicated by the timepiece as a time the occurrence time of the hypoglycemia symptom when it is detected that a predetermined operation is performed based on a signal from the body movement information detection unit; and a communication unit configured to send exercise information comprising the type and time of the exercise performed by the user and hypoglycemia occurrence information comprising the occurrence time of the hypoglycemia symptom, and in which the information processing terminal includes: a receiver configured to receive the exercise information and the hypoglycemia occurrence information from the biological information detector; and a display control unit configured to display an exercise time for each of a plurality of the exercise types on a first display area based on the exercise information, to display an action performed by the user comprising at least one of a medicine-dose, a meal and a sleep in a second display area in a chronological order, and to display a marker in the second display area at a position corresponding to the occurrence time of the hypoglycemia symptom when the hypoglycemia occurrence information is received from the biological information detector.

In the fifth aspect, it is preferable that the exercise information comprises the type of the exercise performed by the user and the exercise time for each of the plurality of exercise types.

In the fifth aspect, it is preferable that the display control unit displays the exercise time for each of the plurality of the exercise types in a graph in the first display area.

### BRIEF DESCRIPTION OF THE DRAWING(S)

FIG. 1 is a schematic illustration showing a biological information measurement system according to an exemplary embodiment of the invention.

FIG. 2 is a block diagram showing an arrangement of a biological information detector according to the exemplary embodiment.

FIG. 3 is a block diagram showing an arrangement of a control unit of the biological information detector according to the exemplary embodiment.

FIG. 4 schematically shows a screen transition on a display according to the exemplary embodiment.

FIG. 5 is a flow chart showing an occurrence timing recordation process for storing the occurrence time of the hypoglycemia symptom in the exemplary embodiment.

FIG. 6 is a block diagram showing an arrangement of an information processor according to the exemplary embodiment.

FIG. 7 is a block diagram showing an arrangement of a controller of the information processor according to the exemplary embodiment.

FIG. 8 shows an example of a food-detail recordation screen according to the exemplary embodiment.

FIG. 9 shows an example of a medicine-dose-detail recordation screen according to the exemplary embodiment.

FIG. 10 shows an example of an exercise result presentation screen according to the exemplary embodiment.

FIG. 11 shows an example of a timing display screen according to the exemplary embodiment.

FIG. 12 shows an example of a summary screen according to the exemplary embodiment.

### DESCRIPTION EXEMPLARY OF EMBODIMENT(S)

Exemplary embodiment(s) of the invention will be described below with reference to the attached drawings.

### Overall Arrangement of Biological Information Measurement System

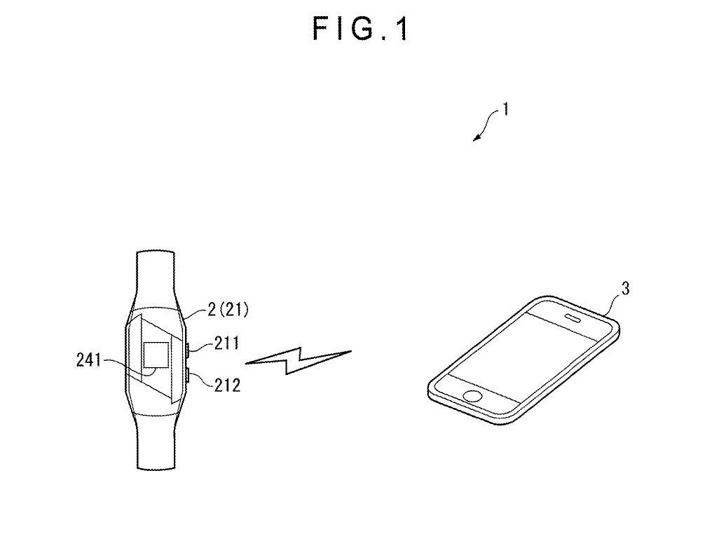

FIG. 1 is a schematic illustration showing a biological information measurement system 1 according to an exemplary embodiment.

As shown in FIG. 1, the biological information measurement system 1 according to the exemplary embodiment includes a biological information detector (sometimes referred to as a detector hereinafter) 2 and an information processor 3.

In the biological information measurement system 1, the detector 2 measures biological information and body movement information. In addition, in response to a predetermined operation, the detection device 2 stores the current date-time as a hypoglycemia occurrence date-time at which a hypoglycemia symptom occurs. The detector 2 then sends date-time information indicating the hypoglycemia occurrence date-time as well as the measured biological information and the body movement information to the information processor 3.

The information processor 3 analyzes an action of a user based on the biological information and the body movement information received from the detector 2. Further, the information processor 3 displays an input screen for inputting food details and medicine-dose details, and stores the food details and the medicine-dose details in response to the input operation by the user. The information processor 3 also displays: a timing display screen including a time table on which the timing of the analyzed action of the user, the mealtime and medicine-dose time, and hypoglycemia symptom occurrence time based on the hypoglycemia occurrence date-time on the date selected by the user are indicated using predetermined markers; and a calendar display screen (summary screen) indicating the date on which the hypoglycemia symptom occurred is indicated using a predetermined marker.

The components of the biological information measurement system 1 will be described below.

### Arrangement of Detector

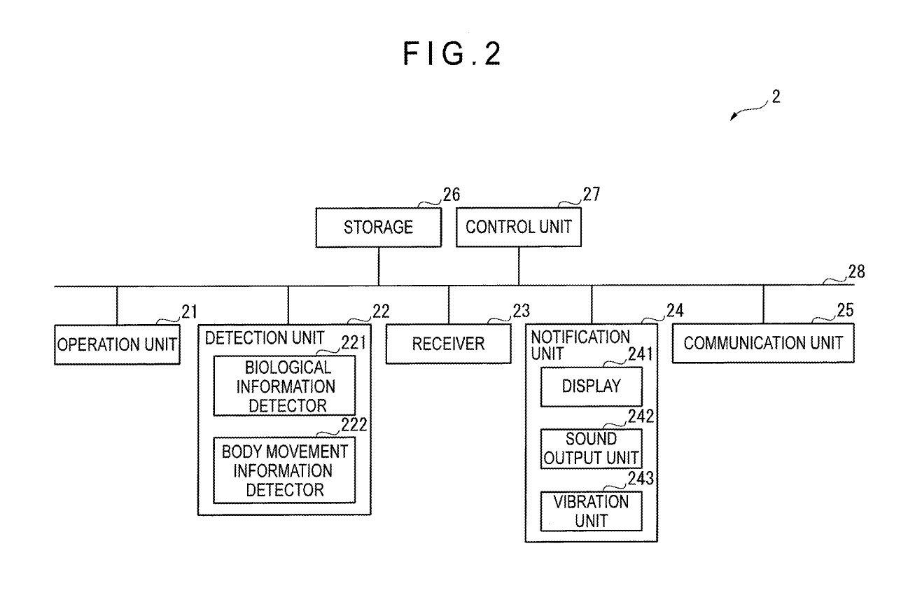

FIG. 2 is a block diagram showing an arrangement of the biological information detector 2 according to the exemplary embodiment.

The detector 2 is a so-called wearable device that is adapted to be worn by a user to detect and store the biological information and the body movement information. The detector 2 stores the current date-time as the hypoglycemia occurrence date-time indicating the time at which the hypoglycemia symptom is occurred when a predetermined operation including a tap operation is performed. In addition, the detector 2 sends the stored biological information, body movement information, and date-time information including the hypoglycemia occurrence date-time to the information processor 3.

As shown in FIG. 2, the detector 2 includes an operation unit 21, a detection unit 22, a receiver 23, a notification unit 24, a communication unit 25, a storage 26 and a controller 27, the components 21 to 27 being electrically connected via a bus-line 28.

### Arrangement of Operation Unit

The operation unit 21 receives an input operation by the user and outputs an operation signal corresponding to the input operation to the controller 27. The operation unit 21 outputs the operation signal corresponding to the input operation on buttons 211, 212 (see FIG. 1) exposed on an exterior of a case of the detector 2 to the controller 27. It should be noted that the operation unit 21 may be configured to recognize a sound (voice) of the user and output an operation signal corresponding to the sound to the controller 27.

Though detailed below, when the user judges himself/herself is in hypoglycemia, the user operates the operation unit 21 (i.e. performs the above predetermined operation) to store the hypoglycemia occurrence date-time in the storage 26.

It should be noted that the operation unit 21 may have a tap operation detection unit. In such an instance, the tap operation is detected based on an acceleration signal detected by an acceleration sensor provided in the tap operation detection unit and the operation signal corresponding to the tap operation may be outputted to the controller 27.

### Arrangement of Detection Unit

The detection unit 22 includes a biological information detection unit 221 configured to detect the biological information of the user and a body movement information detection unit 222 configured to detect the body movement information of the user.

The biological information detection unit 221 is configured to detect the biological information of the user wearing the detector 2. In the exemplary embodiment, the biological information detection unit 221 is configured to detect the biological information in a form of a pulse wave using various sensors. It should be noted that the biological information detection unit 221 may alternatively or additionally be configured to detect biological information in a form of heart rate, blood pressure, body temperature, blood-sugar level, blood-alcohol concentration or the like.

The body movement information detection unit 222 defines an input unit together with the operation unit 21 and is configured to detect body movement information of the user in a form of an acceleration changing in accordance with a body movement of the user The body movement information detection unit 222 is also configured to detect the tap operation of the user based on the change in the acceleration.

### Arrangement of Receiver

The receiver 23 is configured to acquire position information indicating the current position of the detector 2 (i.e. position information indicating the current position of the user). For instance, the receiver 23 is configured to receive electric waves outputted from a satellite of a satellite positioning system such as GPS (Global Positioning System) and the like and acquire the position information indicating the current position based on the electric wave. The receiver 23 outputs the acquired position information to the controller 27. It should be noted that the receiver may alternatively be configured to calculate the position information using a communication wireless electric wave instead of the above arrangement. It should also be noted that the receiver 23 is not necessarily provided.

### Arrangement of Notification Unit

The notification unit 24 notifies various information under the control of the controller 27. The notification unit 24 notifies the user of a working condition of the detector 2, the detected information and the like.

The notification unit 24 includes a display 241, a sound-output unit 242, and a vibration unit 243.

The display 241 is configured to display the above various information using liquid crystal and the like. The display 241 is configured to display a later-described plurality of menu screens and a later-described plurality of setting screens including an occurrence timing recordation screen under the control of the later-described controller 27.

The sound-output unit 242 includes a speaker, and is configured to output sound corresponding to sound information inputted by the controller 27.

The vibration unit 243 includes a motor whose drive is controlled by the controller 27, the condition of the biological information detector 2 being notified using vibrations caused by driving the motor.

### Arrangement of Communication Unit

The communication unit 25 corresponds to a time information transmission unit of the invention, and includes a communication module configured to communicate with an external device such as the information processor 3 through a network. Under the control of the controller 27, the communication unit 25 sends, for instance, the biological information and the body movement information detected by the detection unit 22 and stored in the storage 26 and also sends the date-time information indicating the hypoglycemia occurrence date-time. It should be noted that, though the communication unit 25 wirelessly communicates with a base station or connection device (e.g. a router) connected with a network to be in communication with the information processor 3 in the exemplary embodiment, the connection type and connection form are not limited as long as the communication unit 25 is capable of being in communication with the information processor 3.

### Arrangement of Storage

The storage 26 is provided by a storage device including a flash memory and the like and stores therein a program and data necessary for the operation of the detector 2.

For instance, the data stored in advance in the storage 26 includes connection information for communicating with the information processor 3 using the communication unit 25.

The storage 26 also stores a timing storing program for storing in response to the predetermined operation the current time as the occurrence time of the hypoglycemia symptom.

Further, the storage 26 stores the biological information and the body movement information detected by the detection unit 22 and the position information acquired by the receiver 23 under the control of the controller 27, as well as the date-time information indicating the hypoglycemia occurrence date-time.

### Arrangement of Controller

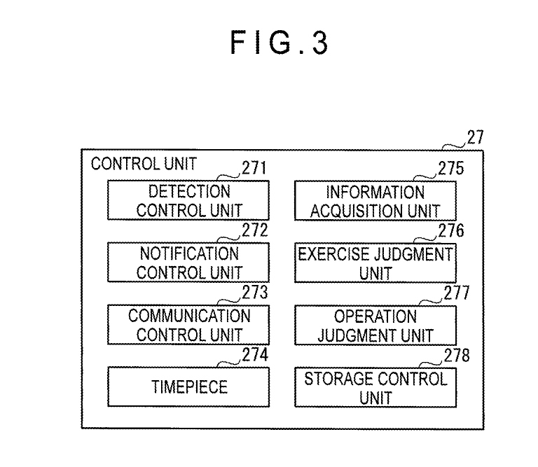

FIG. 3 is a block diagram showing an arrangement of the controller 27.

The controller 27 includes a processing circuit such as a CPU (Central Processing Unit), and controls the operation of the detector 2 in an autonomous manner or in accordance with the operation signal inputted from the operation unit 21 in response to the operation of the user. The controller 27 acquires, for instance, the biological information and the body movement information detected by the detection unit 22 and sends the biological information and the body movement information to the information processor 3. In addition to the above, the controller 27 records the current time as the hypoglycemia occurrence date-time in response to the operation signal corresponding to the predetermined input operation of the user.

The controller 27 includes functional units defined by execution of the program stored in the storage 26 by the processing circuit as shown in FIG. 3, the functional units including a detection control unit 271, a notification control unit 272, a communication control unit 273, a timepiece 274, an information acquisition unit 275, an exercise judgment unit 276, an operation judgment unit 277 and a storage control unit 278.

The detection control unit 271 controls the operation of the detection unit 22 and stores the detection results of the detection unit 22 in the storage 26. The detection control unit 271 commands the detection unit 22 to detect the various information when the detector 2 is worn by the user and the detection unit 22 is capable of detection of the biological information and the body movement information. When the detection unit 22 is unable to detect the information, the detection control unit 271 stops the operation of the detection unit 22. Accordingly, though the detected biological information and the movement information are stored in the storage 26 when the detector 2 is worn by the user, the detected biological information and the movement information are not stored in the storage 26 when the detector 2 is not worn by the user, whereby the consumption power can be restrained.

The notification control unit 272 controls the operation of the notification unit 24. For instance, the notification control unit 272 controls the operation of the display 241 of the notification unit 24 to display the biological information and the body movement information detected by the detection unit 22.

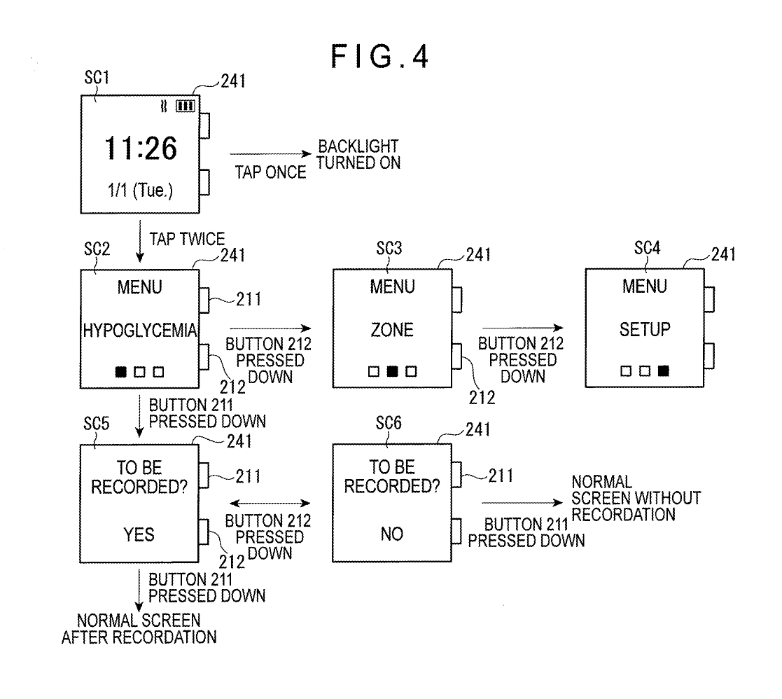

Further, the notification control unit 272 corresponds to the display control unit, which, when it is judged by the later-described operation judgment unit 277 that a tap operation is performed, a menu screen showing a “hypoglycemia” field, from which the screen transitions to the occurrence timing recordation screen, is initially displayed. Then, when it is judged that a button 212 is pressed among the buttons 211, 212, a menu screen showing a “zone” field and a menu screen showing a “setup” field (see FIG. 4) are sequentially displayed each time the button 212 is pressed.

The communication control unit 273 controls the communication unit 25 to establish a communication with the information processor 3 based on the connection information stored in the storage 26.

The timepiece 274 indicates the current date-time.

The information acquisition unit 275 acquires the biological information and the body movement information detected by the detection unit 22 and the position information acquired by the receiver 23 to store the biological information, the body movement information and the position information in the storage 26. At this time, the information acquisition unit 275 stores the detection date-time and acquisition date-time of the biological information, the body movement information and the position information with reference to the current date-time indicated by the timepiece 274 together with the biological information, body movement information and the position information.

The exercise judgment unit 276 analyzes the biological information and the body movement information acquired by the information acquisition unit 275 to judge the type (intensity) of the exercise done by the user.

The type of the exercise done by the user refers to the type of exercise set in advance within a heart rate range (e.g. 80/min to 140/min) classified into a plurality of target heart rate zones (e.g. per 20/min) for exercise that are defined in advance for examinees. For instance, an exercise with the heart rate ranging from 80/min to less than 100/min is classified as a “low-intensity exercise”, an exercise with the heart rate ranging from 100/min to less than 120/min is classified as an “exercise in appropriate zone”, and an exercise with the heart rate ranging from 120/min to less than 140/min is classified as a “high-intensity exercise” by the exercise judgment unit 276 in the exemplary embodiment. The exercise judgment unit 276 thus judges which type of the exercise is done by the user when the user performs an exercise and calculates an exercise time (start time and end time of the exercise) for each of the types of the exercise per one day.

The operation judgment unit 277 judges the type of the input operation done by the user. Specifically, the operation judgment unit 277 judges whether or not the tap operation is done by the user on the detector 2 and whether or not the buttons 211, 212 of the operation unit 21 are pressed by the user.

For instance, the operation judgment unit 277 judges whether or not the tap operation is done based on a presence of a change in the acceleration in accordance with the tap operation in the acceleration signal detected by the body movement information detection unit 222. In other words, the operation judgment unit 277 judges whether or not a signal (acceleration signal) indicating that the tap operation is done is received from the body movement information detection unit 222.

Further, the operation judgment unit 277 judges whether the buttons 211, 212 are pressed based on the operation signal received from the operation unit 21 in response to the input operation (press-down) of the buttons 211, 212, and which one of the buttons 211, 212 is pressed based on the type of the button included in the operation signal.

The storage control unit 278 records the current date-time indicated by the timepiece 274 as the hypoglycemia occurrence date-time at which the hypoglycemia symptom occurs, in response to the input operation of the buttons 211, 212 while a later-described occurrence timing recordation screen SC5 is displayed. Further, the storage control unit 278 stores the biological information and the body movement information respectively detected by the biological information detection unit 221 and the body movement information detection unit 222 in the storage 26.

### Screen Transition of Display

FIG. 4 shows display screen transitions on the display 241.

When no input operation is performed by the user, the notification control unit 272 displays a normal screen SC1 on the display 241 as shown in FIG. 4. The normal screen SC1 includes indications of a remaining battery level and communication status of the detector 2, and the current date-time.

When it is judged by the operation judgment unit 277 that the tap operation is performed once while the normal screen SC1 is displayed, the notification control unit 272 lights a backlight (not shown) of the display 241.

On the other hand, when it is judged by the operation judgment unit 277 that the tap operation is performed consecutively twice while the normal screen SC1 is displayed, the notification control unit 272 displays the menu screen SC2 including the “hypoglycemia” field, which is one of a plurality of items to be displayed and from which the displayed screen transitions to the later-described occurrence timing recordation screen SC5, on the display 241. Other items to be displayed includes “zone” and “setup” fields, where the menu screen SC2 including the “hypoglycemia” field is set preferentially to menu screens SC3, SC4 respectively displaying the “zone” and “setup” fields. Accordingly, when the tap operation is performed consecutively twice, the menu screen SC2 indicating the “hypoglycemia” field is initially displayed.

When it is judged by the operation judgment unit 277 that the button 211 is pressed down while the menu screen SC2 is displayed, the notification control unit 272 displays on the display 241 a selection screen (i.e. the occurrence timing recordation screen SC5) requesting the user's selection on whether or not the current time is to be stored as the hypoglycemia occurrence date-time.

The characters “YES” are displayed on the occurrence timing recordation screen SC5 as an answer for a query “TO BE RECORDED?” When it is judged that the button 212 is pressed down while the occurrence timing recordation screen SC5 is displayed, the notification control unit 272 displays a cancellation screen SC6 showing characters NO instead of “YES” on the display 241. When it is judged that the button 212 is pressed down while the cancellation screen SC6 is displayed, the notification control unit 272 again displays the occurrence timing recordation screen SC5 on the display 241. When the button 211 is pressed, the notification control unit 272 displays the normal screen SC1.

On the other hand, when it is judged that the button 211 is pressed down while the occurrence timing recordation screen SC5 is displayed, the storage control unit 278 records the current time as the hypoglycemia occurrence date-time. Subsequently, the notification control unit 272 displays the normal screen SC1 on the display 241.

The two consecutive tap operations as described above correspond to a first operation medicine-dose, whereas the input operation on the button 211 corresponds to a second operation medicine-dose. In other words, the predetermined operation is a combination of the two consecutive tap operations corresponding to the first operation and the input operation on the button 211 corresponding to the second operation.

It should be noted that, when it is judged that the button 212 is pressed down while the menu screen SC2 is displayed, the notification control unit 272 displays the menu screen SC3 on the display 241. When it is judged that the button 211 is pressed down while the menu screen SC3 is displayed, the screen transitions to a zone setting screen (not illustrated) for setting the zone.

Further, when it is judged that the button 212 is pressed down while the menu screen SC3 is displayed, the notification control unit 272 displays the menu screen SC4 on the display 241. When it is judged that the button 211 is pressed down while the menu screen SC4 is displayed, the screen transitions to a setup screen (not illustrated) for inputting user information (e.g. body height and weight).

On the other hand, when it is judged that the button 212 is pressed down while the menu screen SC4 is displayed, the notification control unit 272 again displays the menu screen SC2 on the display 241.

As described above, when the tap operation is performed consecutively twice by the user, the notification control unit 272 displays the menu screen SC2 including the “hypoglycemia” field on the display 241 in preference to the other menu screens SC3, SC4 from among the plurality of menu screens.

### Timing Recordation Process

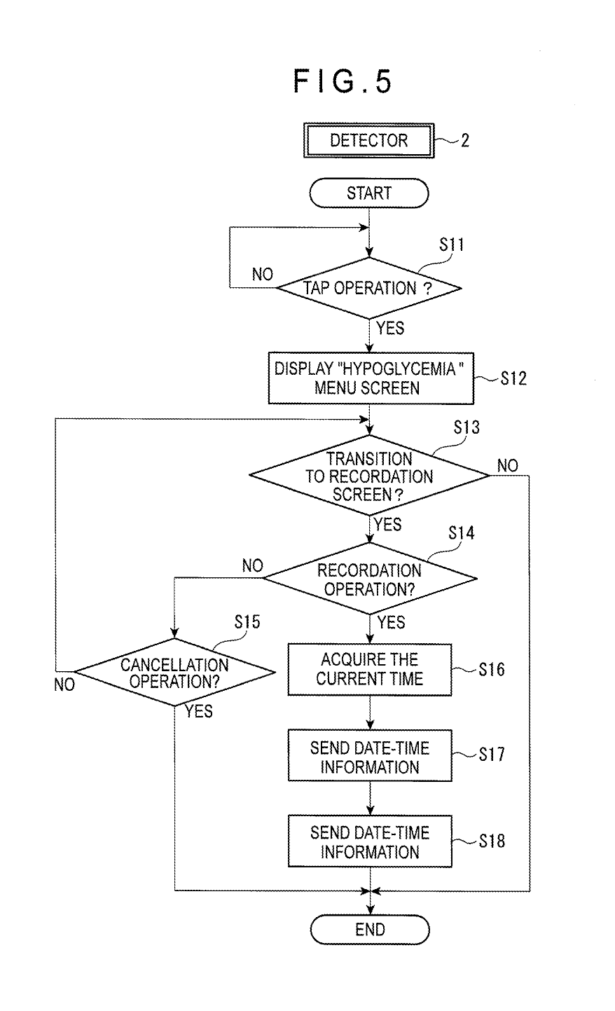

FIG. 5 is a flow chart showing an occurrence timing recordation process for recording the occurrence timing of the hypoglycemia symptom performed by the biological information detector 2.

As described above, the detector 2 performs the timing recordation process, in which the hypoglycemia occurrence date-time at which the hypoglycemia symptom occurs is stored in response to the input operation by the user and the date-time information indicating the hypoglycemia occurrence date-time is sent to the information processor 3. On the other hand, the information processor 3 stores the received date-time information in the storage 35.

In the timing recordation process, as shown in FIG. 5, the operation judgment unit 277 of the detector 2 judges whether or not the two consecutive tap operations are performed based on the acceleration signal received from the body movement information detection unit 222 (Step S11).

When it is judged that the two tap operations are not performed in the judgment process in Step S11, the controller 27 returns the timing recordation process to the process of Step S11.

On the other hand, when it is judged in the judgment process of Step S11 that the two tap operations are performed, the notification control unit 272 displays the menu screen SC2 indicating the “hypoglycemia” field on the display 241 (Step S12).

Then, it is judged whether or not the screen of the display 241 has transitioned from the menu screen SC2 showing the “hypoglycemia” field to the occurrence timing recordation screen SC5 (Step S13). Specifically, the operation judgment unit 277 judges whether or not the button 211 is pressed while the menu screen SC2 indicating the item “hypoglycemia” field is displayed on the display 241.

When it is judged that the screen of the display 241 has not transitioned to the occurrence timing recordation screen SC5 in the judgment process of Step S13, the controller 27 terminates the process.

On the other hand, when it is judged that the screen of the display 241 has transitioned to the occurrence timing recordation screen SC5 in the judgment process of Step S13, the operation judgment unit 277 judges whether or not the recordation operation is performed (Step S14). Specifically, the operation judgment unit 277 judges whether or not the button 211 is pressed while the occurrence timing recordation screen SC5 is displayed on the display 241.

When it is judged that the recordation operation is not performed in the judgment process in Step S14, the operation judgment unit 277 judges whether or not a cancellation operation is performed (Step S15). Specifically, the operation judgment unit 277 judges whether or not the button 212 is pressed while the occurrence timing recordation screen SC5 is displayed on the display 241 and whether or not the button 211 is further pressed while the cancellation screen SC6 is displayed on the display 241.

When it is judged in the judgment process in Step S15 that the cancellation operation is not performed, the controller 27 returns the timing recordation process to the process of Step S13.

On the other hand, when it is judged in the judgment process in Step S15 that the cancellation operation is performed, the controller 27 terminates the process.

On the other hand, when it is judged in the judgment process in Step S14 that the recordation operation is performed, the storage control unit 278 acquires the current date-time indicated by the timepiece 274 (Step S16). Then, the storage control unit 278 records the acquired current date-time as the hypoglycemia occurrence date-time (date-time information) at which the hypoglycemia symptom occurred in the storage 26 (Step S17).

Then, the communication control unit 273 sends the biological information, the body movement information, the position information and the date-time information stored in the storage 26 to the information processor 3 at a predetermined time interval (e.g. once in twenty minutes) (Step S18).

The timing recordation process thus ends. It should be noted that the information processor 3 stores the received information.

### Arrangement of Information Processor

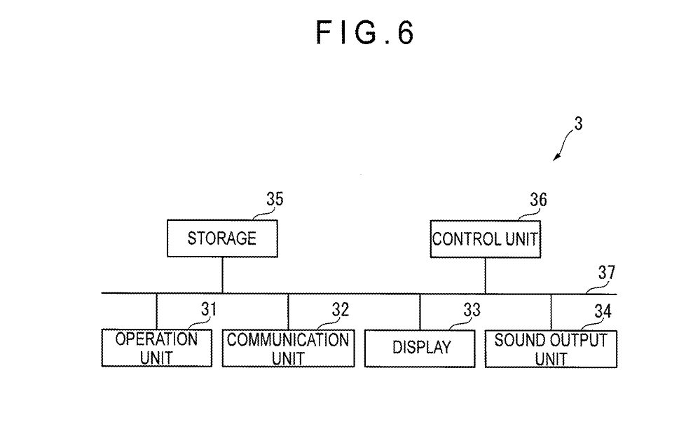

FIG. 6 is a block diagram showing an arrangement of the information processor 3.

The information processor 3 is provided by, for instance, a smartphone (multifunctional portable cellular phone), a tablet, Personal Computer (PC) and the like.

The information processor 3 stores and manages the biological information, the body movement information, the position information and the date-time information received from the detector 2 as described above. In addition, the information processor 3 analyzes the biological information, the body movement information, the position information and the date-time information to calculate and store, for instance, start and end times of an exercise and exercise time. Further, the information processor 3 displays a recordation screen for recording information on food and medicine-dose, and records the food details and the medicine-dose details inputted by the user. Further, the information processor 3 displays the later-described timing display screen to indicate the timing for exercise, meal and medicine-dose, and the occurrence time of the hypoglycemia. In addition, the information processor 3 displays the later-described calendar display screen (summary screen) to indicate the occurrence date of the hypoglycemia.

As shown in FIG. 6, the information processor 3 includes an operation unit 31, a communication unit 32, a display unit 33, a sound output unit 34, a storage 35, and a control unit 36, which are mutually connected via a bus-line 37.

### Arrangement of Operation Unit

The operation unit 31 receives an input operation by the user and outputs operation information corresponding to the input operation to the control unit 36. The operation unit 31 is, for instance, provided by a physical key or a touch panel provided to a case of the information processor 3, or by a keyboard, pointing device or the like connected to the information processor 3 by wired or wireless transmission.

### Arrangement of Communication Unit

The communication unit 32 includes a first communication module capable of communication with an external device such as the detector 2, and a second communication module capable of communication with a server (not illustrated) on the network such as the Internet. The communication unit 32 communicates with the external device and the server under the control of the control unit 36. It should be noted that, when the communication unit 32 is capable of communication with the external device and the server using the same communication system, it is only necessary for the communication unit 32 to have one of the first and second communication modules. For instance, when it is not necessary to communicate with the server, the second communication is not necessarily provided.

### Arrangement of Display and Sound-Output Unit

The display unit 33 corresponds to a processor-side display in the exemplary embodiment. The display unit 33 is provided by, for instance, a liquid crystal panel, an organic electro-luminescence (EL) panel or an electrophoretic display panel to display an image generated by a later-described presentation information generation unit 368. Specifically, the display unit 33 displays an execution screen (e.g. a later-described execution screen ES) of an OS (Operating System) and various applications run by the control unit 36.

The sound output unit 34 includes a speaker, and is configured to output sound corresponding to sound information inputted by the control unit 36. For instance, the sound output unit 34 outputs sound corresponding to the information to be presented to the user when the control unit 36 executes a later-described information management application.

### Arrangement of Storage

The storage 35 is provided by a storage device such as an SSD (Solid State Drive), HDD (Hard Disk Drive) and flash memory and stores program(s) and data required for the operation of the information processor 3. Such a program stored in the storage 35 include the OS for controlling the information processor 3 and the later-described information management application.

The storage 35 also stores the various information received from the detector 2 and the input data inputted on later-described recordation screens.

### Arrangement of Control Unit

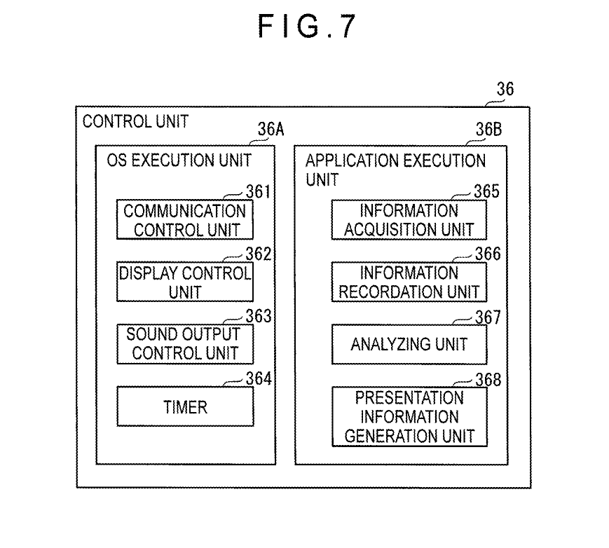

FIG. 7 is a block diagram showing an arrangement of the control unit 36.

The control unit 36 includes a CPU (Central Processing Unit) and executes the program stored in the storage 35 to control the operation of the information processor 3. The control unit 36 includes an OS execution unit 36A and an application execution unit 36B.

The OS execution unit 36A is a functional unit configured to run the OS stored in the storage 35. The OS execution unit 36A includes a communication control unit 361, a display control unit 362, a sound-output control unit 363 and a timepiece 364.

The communication control unit 361 is configured to control the communication unit 32 to communicate with the external device and the server.

The display control unit 362 displays the above execution screen and the execution screen (an execution screen generated by other component) of the other application and OS on the display unit 33.

The sound-output control unit 363 outputs to the sound output unit 34 sound information of a sound to be outputted when the OS and the application(s) are executed.

The timepiece 364 indicates the current date-time.

The application execution unit 36B runs one of the applications stored in the storage 35 and commanded by the OS execution unit 36A in response to the operation information inputted by the operation unit 31.

The application execution unit 36B includes an information acquisition unit 365, an information recordation unit 366, an analyzing unit 367 and the presentation information generation unit 368 that function when corresponding one of the information management applications stored in the storage 35 is run.

The information acquisition unit 365 acquires the information inputted by the user on the execution screen (recordation screen of food details and medicine-dose details) of the later-described information management application. In addition, the information acquisition unit 365 acquires the various information (the biological information, the body movement information, the position information and the date-time information) from the detector 2 through the communication unit 32.

The information recordation unit 366 records the information acquired by the information acquisition unit 365 in the storage 35.

The analyzing unit 367 analyzes the biological information, the body movement information, the position information and the date-time information received from the detector 2 to generate analysis results including consumed calorie, number of footsteps and exercise time (estimated start and end time of the user). In addition, the analyzing unit 367 judges sleep-onset time and wake-up time of the user based on the biological information, the body movement information and the position information and generates analysis results including the sleep period and the like of the user.

The presentation information generation unit 368 corresponds to the timing display screen generation unit and the calendar display screen generation unit and generates the execution screen of the information management application. For instance, the presentation information generation unit 368 generates the exercise result presentation screen (execution screen) based on the analysis results of the analyzing unit 367. Further, the presentation information generation unit 368 generates the recordation screen (execution screen) for the meal record and medicine-dose record. The screens are displayed on the display unit 33 under the control of the display control unit 362. Further, the presentation information generation unit 368 displays the timing display screen to indicate the time for exercise, mealtime, the time for medicine-dose, and the occurrence time of the hypoglycemia using markers. In addition, the presentation information generation unit 368 displays the later-described calendar display screen (summary screen) to indicate the occurrence date of the hypoglycemia.

The items generated by the presentation information generation unit 368 will be described in detail later.

### Arrangement of Execution Screen

The display control unit 362 displays the following execution screens ES (ES1 to ES5) on the display unit 33 based on the processing results of the application execution unit 36B when the information management application is run by the application execution unit 36B.

The components and functions of the execution screens ES will be described below.

### Food-Detail Recordation Screen

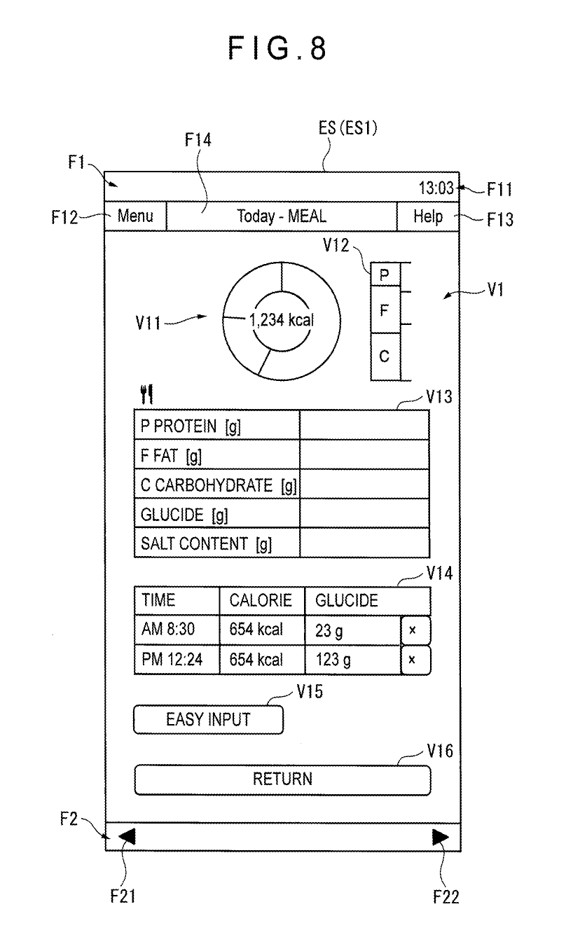

FIG. 8 shows an example of a food-detail recordation screen ES1.

The food-detail recordation screen ES1 is a part of the execution screen ES, in which the food taken in by the user on a selected date (the current day by default) is recorded and displayed. As shown in FIG. 8, the food-detail recordation screen ES1 includes fixed display areas F1, F2 respectively on upper and lower sides of the screen, and variable display area V1 interposed between the fixed display areas F1, F2.

A time display area F11 that is configured to show the current time indicated by the timepiece 364 is disposed on an upper field of the fixed display area F1 on the upper side of the screen. A button F12 for transition to a menu screen (not shown) once being pressed (input) is disposed on the left side in the lower field of the fixed display area F1. A button F13 for transition to a help screen (not shown) once being pressed is disposed on the right side in the lower field of the fixed display area F1. A title F14 showing the content of the screen is disposed in an area between the buttons F12, F13.

Buttons F21, F22 are respectively disposed on the left and right sides of the fixed display area F2 on the lower side of the screen. The buttons F21, F22 are used for screen transition.

A total calorie intake display field V11, a intake ratio display field V12 showing an intake ratio of protein (P), fat (F) and carbohydrate (C), a display field V13 showing food details, a display field V14 showing a record of the food, an easy-input button V15, and a return button V16 are disposed in the variable display area V1.

The total calorie intake display field V11 shows a total calorie intake on the current date at the center of a circle graph. The intake ratio of protein (P), fat (F) and carbohydrate (C) in the meal of the day is displayed in the intake ratio display field V12 in a bar graph.

The intake amount (g) of protein (P), fat (F), carbohydrate (C), glucide and salt content are displayed in the display field V13.

In addition, the record of the meal of the day is displayed in the display field V14. The record of the meal includes the start time, the intake calorie and the glucide amount of the meal.

Though not illustrated, when the easy-input button V15 is pressed down, a screen for recording the start time, the type and the amount of the meal (i.e. subjective and relative information on the amount of the meal) is displayed. The user selects the type of the meal (e.g. “meal”, “alcohol drinking” and “snack”) and sets the amount of food taken in the selected type of the meal on the screen. With the above arrangement, the intake amount of protein, fat, carbohydrate, glucide and salt content in each of the meals and the total calorie of the meal are roughly calculated. It should be noted the details of the meal may be analyzed with reference to an external database (database stored in an external device) or may be analyzed through an external server (external device).

When the return button V16 is pressed, the screen transitions to a screen displayed immediately before the food-detail recordation screen ES1 is displayed.

### Medicine-Dose-Detail Recordation Screen

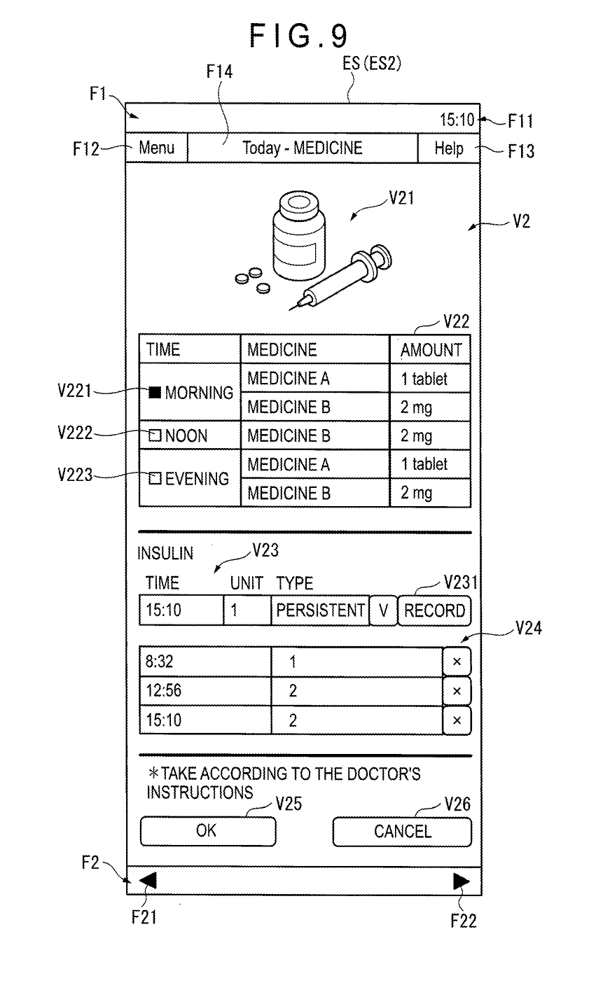

FIG. 9 shows an example of a medicine-dose-detail recordation screen ES2.

The medicine-dose-detail recordation screen ES2 is a part of the execution screen ES, in which the user inputs the medicine-dose details. As shown in FIG. 9, the medicine-dose-detail recordation screen ES2 includes the fixed display areas F1, F2 and the variable display area V2.

An image screen V21 showing that the displayed screen is used for recordation of the medicine-dose detail, an medicine-taking schedule field V22 showing medicine-taking schedule prescribed by a doctor-in-charge in advance, an input field V23 on which medicine-taking time of insulin is designed to be input, a display field V24 indicating the time at which insulin is administered, an OK button V25 and a cancel button V26 are provided in the variable display area V2.

The medicine-taking schedule field V22 includes “time”, “medicine” and “amount” fields, where, for instance, a schedule of administration of 1 pill of “medicine A” and 2 mg of “medicine B” in the morning and evening, and a schedule of administration of 2 mg of “medicine B” in the noon are set. In the “time” field, check fields V221, V222, V223 to be checked by the user when the user takes the “medicine A” and “medicine B” are set for each of items of morning, noon and evening. When the user touches the check fields V221, V222 and V223, as shown, for instance, in the check field V221, the color of the check field V221 changes. Accordingly, the user can easily understand by viewing the check fields V221, V222, V223 whether or not the medicine is taken as scheduled.

Selection fields capable of selecting the time of dose of insulin, dosed amount (unit) and type (e.g. persistent or fast-acting) and a recordation button V231 are provided in the input field V23. It should be noted that the current time is displayed by default in the selection field for selecting the medicine-taking time. When the recordation button V231 is pressed after the medicine-dose details are inputted in each of the selection fields in the input field V23 by the user, the medicine-dose details are added in the display field V24.

The display field V24 is a table in which the medicine-dose details of insulin are displayed. The display field V24 includes a plurality of rows in which the time of dose and dosed amount (unit) of insulin recorded in the input field V23 are displayed. In addition, each of the rows of the input field V23 includes a button for deleting the inputted medicine-dose details.

When the OK button V25 is pressed, the selected state of the check fields V221, V222, V223 (i.e. medicine-taking details), and medicine-dose details displayed in the display field V24 is fixed, and the information acquisition unit 365 records the information in the storage 35. On the other hand, when the cancel button V26 is pressed, the changed medicine-taking details and the medicine-dose details are cancelled.

### Exercise Result Presentation Screen

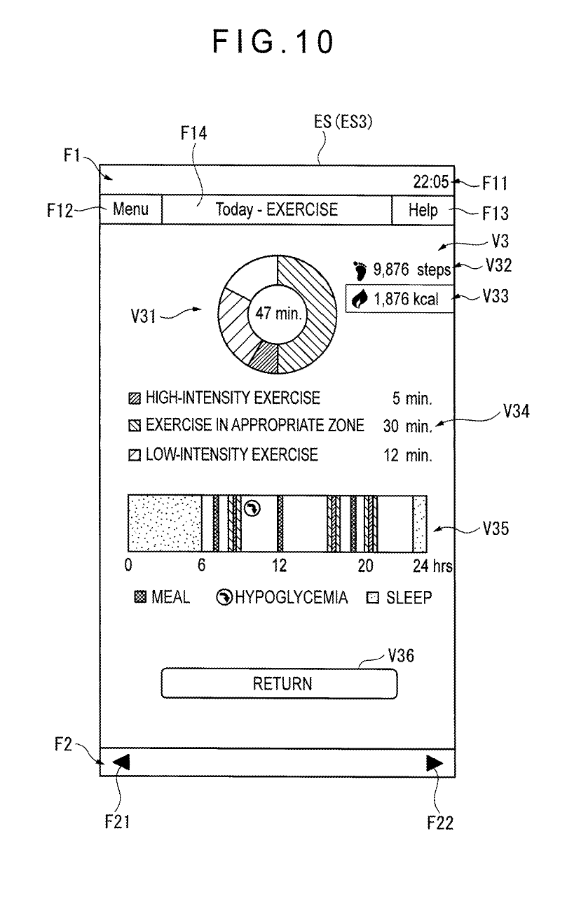

FIG. 10 shows an example of an exercise result presentation screen ES3.

The exercise result presentation screen ES3 is a part of the execution screen ES, where the analysis results of the exercise based on the biological information and the body movement information acquired from the detector 2, as well as the timing of the exercise and meal within a predetermined time period (the date selected by the user in the exemplary embodiment) are presented. As shown in FIG. 10, the exercise result presentation screen ES3 includes the fixed display areas F1, F2 and the variable display area V3 interposed between the fixed display areas F1, F2.

A total exercise time display area V31, a footsteps display area V32, a consumed calorie display area V33, a detailed exercise time display area V34 and a timing display area V35 indicating the information on the selected date and a return button V36 are provided in the variable display area V3.

The total exercise time display area V31, the footsteps display area V32 and the consumed calorie display area V33 respectively display a total exercise time, the number of footsteps and the consumed calorie on the selected date. Among the above areas, the total exercise time display area V31 includes a circle graph showing the total exercise time on the selected date at the center thereof, the circle graph showing the exercise time of each of the “high-intensity exercise”, “exercise in appropriate zone” and “light exercise” occupied in the total exercise time.

The detailed exercise time display area V34 shows the exercise time for each of the exercise categories on the date.

A time table showing the exercise period, mealtime and sleep time on the date correspondingly to the time in the predetermined time period is displayed in the timing display area V35. It should be noted that, when the above date-time information indicating the date has been acquired, a mark (active time marker) indicating the timing of the exercise, meal and medicine-dose and a mark (hypoglycemia occurrence marker) showing the occurrence time of the hypoglycemia symptom are displayed on the time table at the time corresponding to the date-time information.

When the return button V36 is pressed, the screen transitions to a screen displayed immediately before the exercise result presentation screen ES3 is displayed.

### Timing Display Screen

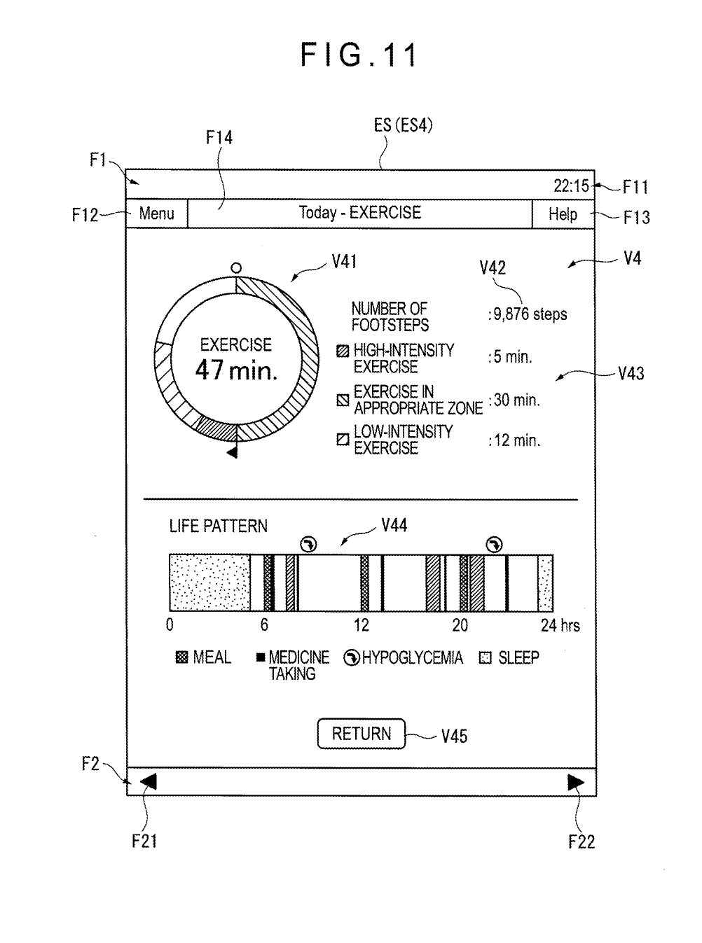

FIG. 11 shows an example of a timing display screen ES4.

The timing display screen ES4 is a part of the execution screen ES Similar to the above exercise result presentation screen ES3, the timing display screen ES4 presents the analysis results of the exercise on the date selected by the user, the period of exercise, meal and medicine-dose and the occurrence time of the hypoglycemia symptom. As shown in FIG. 11, the timing display screen ES4 includes the fixed display areas F1, F2 and the variable display area V4 interposed between the fixed display areas F1, F2.

A total exercise time display area V41, a footsteps display area V42, a detailed exercise time display area V43, a timing display area V44 and a return button V45 are provided in the variable display area V4.

Among the above areas, the same contents as those of the total exercise time display area V31, the footsteps display area V32 and the detailed exercise time display area V34 in the exercise result presentation screen ES3 are respectively displayed in the total exercise time display area V41, the footsteps display area V42 and the detailed exercise time display area V43.

A time table from 0 to 24 o'clock time is displayed in the timing display area V44 in the same manner as the timing display area V35. The time table shows the exercise period, mealtime and sleep time on the date selected by the user in accordance with the time set for the time table. Additionally, a mark (active time marker) indicating the time of the exercise, meal and medicine-dose and, when the above date-time information indicating the date has been acquired, a mark (hypoglycemia occurrence marker) showing the occurrence time of the hypoglycemia symptom are displayed on the time table at the corresponding time. The action of the user prior to the occurrence time of the hypoglycemia symptom can be easily recognized by checking the timing display area V44.

When the return button V45 is pressed, the screen transitions to a screen displayed immediately before the timing display screen ES4 is displayed.

### Summary Screen

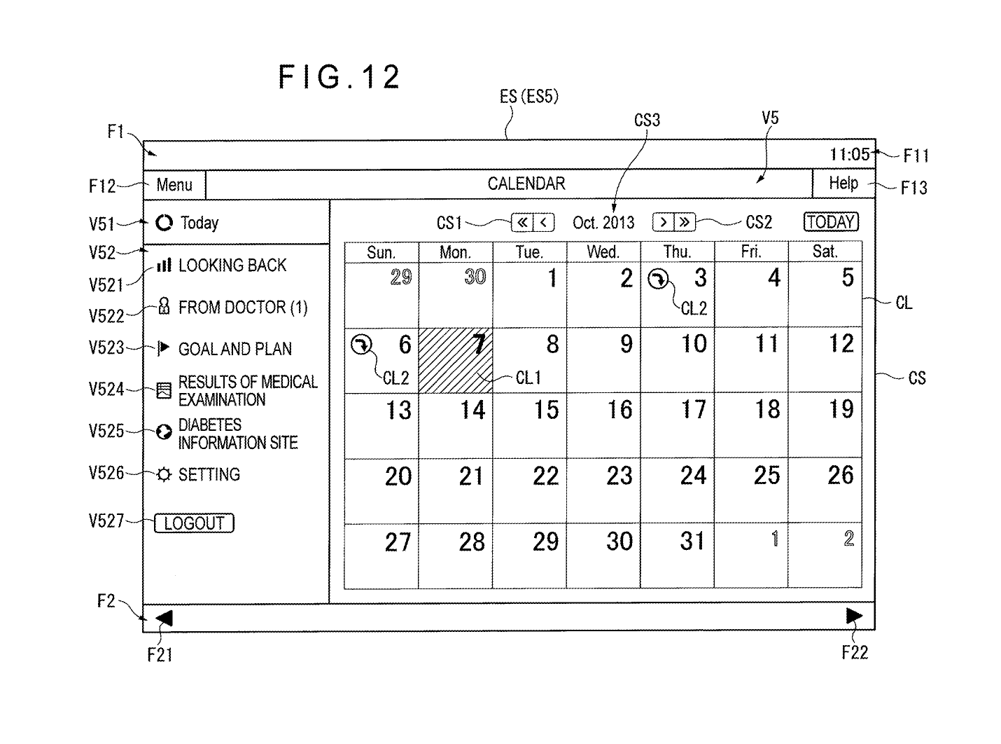

FIG. 12 shows an example of a screen ESS.

The summary screen ES5 is a part of the execution screen ES. The summary screen ES5 includes a calendar of the “month” selected by the user and a mark indicating the date on which the hypoglycemia symptom occurred on the calendar. As shown in FIG.

12, the summary screen ES5 includes the fixed display areas F1, F2 and the variable display area V5 interposed between the fixed display areas F1, F2. It should be noted that a title indicating the contents of the screen corresponding to the menu displayed in later-described menu display area V52 is displayed on the fixed display area F1 of the summary screen ES5 at the position of the title F14 indicating the contents of the screen.

A button V51, a menu display area V52 and a display area CS are provided in the variable display area V5.

The button V51 is used for communication with the detector 2 to acquire various information from the detector 2.

A plurality of buttons V521 to V526 for switching the display contents in the display area CS, and a log-out button V527 are displayed instead of the title F14 in the menu display area V52. When the button V521 is pressed, the display contents on the display area CS is switched to a later-described calendar CL. When the button V522 is pressed, the display contents on the display area CS is switched to a screen displaying a message from a doctor-in-charge. When the button V523 is pressed, the display contents on the display area CS is switched to a screen for checking and/or modifying the goal and plan of the user. When the button V524 is pressed, the display contents on the display area CS is switched to a screen for checking the results of a medical examination of the user acquired from the server. When the button V525 is pressed, the display contents on the display area CS is switched to a screen of diabetes information site acquired from the server. When the button V526 is pressed, the display contents on the display area CS is switched to a setting screen for performing various setting. When the log-out button V527 is pressed, the summary screen ES5 is turned off.

When the calendar CL shown in FIG. 12 is displayed in the display area CS, the summary screen ES5 defines the calendar display screen. The calendar CL includes “day” fields belonging to the “month” selected by the user and arranged in each “week” group. A button CS1 for transition to the calendar CL of preceding month is disposed on the upper left side of the calendar CL in the display area CS. A button CS2 for transition to the calendar CL of the next month is disposed on the upper right side of the calendar CL in the display area CS. A month-display area CS3 showing the “month” of the calendar CL is disposed in an area between the buttons CS1, CS2.

One of the date fields representing the current date (e.g. 7th day) in the calendar CL is provided with an indication CL1 (e.g. color) for distinction from the other date fields. Further, the other of the date fields indicated by the above date-time information (i.e. the date on which the hypoglycemia symptom occurred) (e.g. 3rd and 6th days) is provided with a hypoglycemia occurrence date marker CL2 indicating the occurrence of the hypoglycemia symptom.

The date on which the hypoglycemia symptom occurred can be easily recognized by checking the calendar CL.

When the log-out button V527 is pressed, the screen transitions to a screen displayed immediately before the summary screen ES5 is displayed.

### Advantage(s) of Exemplary Embodiment(s)

The biological information measurement system 1 described above provides the following advantages.

According to the biological information measurement system 1 according to the above-described exemplary embodiment, the date-time indicated by the timepiece 274 when the two consecutive tap operations and the input operation (predetermined operation) are made by the user is stored in the storage 26 as the occurrence time of the hypoglycemia symptom. Accordingly, the user can record the occurrence time of the hypoglycemia symptom only by performing the two consecutive tap operations and the input operation on the button 211. Thus, the cause of the hypoglycemia symptom can be studied based on the recorded time (date-time information) and the biological information at the recorded time.

Since the occurrence time of the hypoglycemia symptom cannot be recorded without the combination of the two different operations including the first operation (the two consecutive tap operations) and the second operation (the input operation on the button 211), erroneous recordation of the time (date-time information) can be restrained.

Since the time (date-time information) is recorded as the occurrence time of the hypoglycemia symptom when the two consecutive tap operations and the input operation on the button 211 are performed by the user, the erroneous recordation of the above time can be reliably restrained.

Since the menu screen SC2 showing the “hypoglycemia” field for transition to the occurrence timing recordation screen SC5 for recording the above time is displayed when the consecutive two tap operations are performed, the user can easily recognize that the above time (date-time information) can be recorded by performing the input operation on the button 211 while the menu screen SC2 is displayed. Accordingly, the recordation operation is facilitated.

When the consecutive two tap operations are performed, the menu screen SC2 having the “hypoglycemia” field for the transition to the occurrence timing recordation screen SC5 in a manner preferential to the other items (“zone” and “setup” fields) among the plurality of items (“hypoglycemia”, “zone” and “setup” fields) is displayed. Accordingly, the user can record the above time by performing the input operation on the button 211 while the menu screen SC2 is displayed. Accordingly, the operation for recording the above time can be rapidly performed.

Since the storage control unit 278 records the biological information and the body movement information in the storage 26, the study on the cause of the hypoglycemia symptom can be more easily made using not only the biological information detected by the biological information detection unit 221 but also the body movement information detected by the body movement information detection unit 222.

Since the information processor 3 records the time information (date-time information) indicating the above time and time of at least one of the user's meal, exercise and medicine-dose inputted by the user, a more detailed study on the cause of the hypoglycemia symptom can be made based on the recorded time of the occurrence of the hypoglycemia symptom and the time of the at least one of meal, exercise and medicine-dose.

Since the hypoglycemia occurrence marker in accordance with the time information on the occurrence of the hypoglycemia symptom and the active time marker in accordance with the time of the meal, exercise and medicine-dose are displayed on a time table showing the time within a predetermined time band in a chronological order in the timing display screen (timing display area V44), the occurrence time of the hypoglycemia symptom, the mealtime, exercise time and medicine-dose time can be more easily recognized by the user.

Since the summary screen ES5 including the calendar CL in which the hypoglycemia occurrence marker is disposed in accordance with the date included in the date-time information on a calendar having the date fields within a predetermined time period arranged in a chronological order is displayed, the date on which the hypoglycemia symptom occurred can be easily recognized by the user.

In the exemplary embodiment, the current time can be recorded as the occurrence time of the hypoglycemia symptom only by performing the predetermined input operations (two consecutive tap operations and input operation on the button 211). Accordingly, the user can record the occurrence time of the hypoglycemia symptom without a troublesome work (e.g. taking a note).

### Modification(s)

The scope of the invention is not limited to the above-described exemplary embodiment, but includes modification(s), improvement(s) and the like as long as the modification(s), improvement(s) and the like are compatible with the objective of the invention.

In the exemplary embodiment, the timepiece 274 indicates the current date-time. However, the scope of the invention is not limited thereto. In the exemplary embodiment, the timepiece 274 may only time the current time. In such an arrangement, it is only necessary that the biological information and the body movement information detected by the detection unit 22, the position information acquired by the receiver 23, and the time information stored in the storage 26 are sent to the information processor 3 as necessary.

In the exemplary embodiment, the time indicated by the timepiece 274 is recorded in the storage 26 as the occurrence time of the hypoglycemia symptom when the user performs the combination of different operations (the predetermined operation) including the two consecutive tap operations and the input operation on the button 211. However, the scope of the invention is not limited thereto. For instance, the predetermined operation is not necessarily the combination of the different operations. Specifically, the predetermined operation may alternatively be an alternate pressing of the buttons 211 and 212, or may alternatively be consecutive four tap operations. In other words, the predetermined operation may be any kind of operation.

In the exemplary embodiment, the time indicated by the timepiece 274 is recorded in the storage 26 as the occurrence time of the hypoglycemia symptom when the input operation on the button 211 is performed after the two consecutive tap operations by the user. However, the scope of the invention is not limited thereto. For instance, the time indicated by the timepiece 274 may be recorded in the storage 26 as the occurrence time of the hypoglycemia symptom when the two consecutive tap operations are performed after the input operation on the button 211.

In the exemplary embodiment, the time indicated by the timepiece 274 is recorded in the storage 26 as the occurrence time of the hypoglycemia symptom when the input operation on the button 211 is performed while the menu screen SC2 including the “hypoglycemia” field for transition to the occurrence timing recordation screen SC5 is displayed on the display 241 after the two consecutive tap operations by the user. However, the scope of the invention is not limited thereto. For instance, the occurrence timing recordation screen SC5 may be displayed after the two consecutive tap operations are performed by the user. In this arrangement, the time indicated by the timepiece 274 can be recorded in the storage 26 as the occurrence time of the hypoglycemia symptom only by pressing the button 211 once after the two consecutive tap operations.

In the exemplary embodiment, the menu screen SC2 including the “hypoglycemia” field, which is preferential to the menu screens SC3, SC4 respectively including the “zone” field and the “setup” field, is displayed when the two consecutive tap operations are performed by the user. However, the scope of the invention is not limited thereto. For instance, it is only necessary that the “hypoglycemia” field is preferential to the other of the plurality of fields even when a plurality of fields are listed on a single menu screen. In other words, the number of the menu screen may be one.

Alternatively, the menu screens SC3, SC4 respectively including the “zone” field and the “setup” field in the exemplary embodiment may be preferential to the menu screen SC2 including the “hypoglycemia” field. With such an arrangement, for instance, the convenience for a user who has no hypoglycemia symptom can be enhanced.

In the exemplary embodiment, the detection unit 22 includes the body movement information detection unit 222. However, the scope of the invention is not limited thereto. For instance, the detection unit 22 may be provided solely with the biological information detection unit 221.

In the exemplary embodiment, the display 241 is provided. However, the scope of the invention is not limited thereto. For instance, the display 241 is not necessarily provided. In this case, it is only necessary that the time indicated by the timepiece 274 is recorded in the storage 26 as the occurrence time of the hypoglycemia symptom when the two consecutive tap operations and the input operation on the button 211 are performed by the user.

In the exemplary embodiment, the information processor 3 includes the presentation information generation unit 368 that generates the timing display screen ES4 and the display control unit 362 that displays the timing display screen ES4 on the display unit 33. However, the scope of the invention is not limited thereto. For instance, the presentation information generation unit 368 may not necessarily generate the timing display screen ES4. In other words, it is only necessary in the exemplary embodiment that the occurrence time of the hypoglycemia symptom and one of the time of at least one of meal, exercise and medicine-dose are stored.

In the exemplary embodiment, the information processor 3 includes the presentation information generation unit 368 that generates the calendar display screen (the summary screen ES5 including the calendar CL) and the display control unit 362 that displays the calendar display screen on the display unit 33. However, the scope of the invention is not limited thereto. For instance, the presentation information generation unit 368 may not necessarily generate the summary screen ES5.

Alternatively, it is possible that the presentation information generation unit 368 does not generate the timing display screen ES4 and the summary screen ES5, but a server including an information generation unit corresponding to the presentation information generation unit 368 is connected to the information processor 3 so that the server acquires the screens ES4, ES5 and displays the screens ES4, ES5 on the display unit 33.

Further, the screens ES4, ES5 generated by the server are not necessarily acquired by the information processor 3 but may be acquired by the other information processor having a display on which the screens are displayed.

In the exemplary embodiment, the exercise judgment unit 276 of the controller 27 is provided to the detector 2. However, the scope of the invention is not limited thereto. For instance, the exercise judgment unit 276 may be provided to the controller 36 of the information processor 3. Alternatively, the same processing as that of the exercise judgment unit 276 may be performed by the analyzing unit 367.

In the exemplary embodiment, the time indicated by the timepiece 274 is simply stored in the storage 26 as the occurrence time of the hypoglycemia symptom when the two consecutive tap operations and the input operation on the button 211 are performed by the user. However, the scope of the invention is not limited thereto. For instance, a predetermined sound may be outputted by the sound-output unit 242 or the vibration unit 243 may be vibrated when the time is stored in the storage 26.

In the exemplary embodiment, the process of Step S16 is performed in the timing recordation process of the detector 2 to send the date-time information to the information processor 3. However, the scope of the invention is not limited thereto. For instance, the Step S16 is not necessarily performed. In this case, the date-time information stored in the storage 26 of the detector 2 may be acquired together with the biological information and the like when the detector 2 is connected with a cradle device.

In the exemplary embodiment, an exercise with the heart rate ranging from 80/min to 100/min is classified as a “low-intensity exercise”, an exercise with the heart rate ranging from 100/min to 120/min is classified as an “exercise in appropriate zone,” and an exercise with the heart rate ranging from 120/min to 140/min is classified as a “high-intensity exercise” by the exercise judgment unit 276. However, the scope of the invention is not limited thereto. In other words, the heart rate range may be defined as desired. For instance, the heart rate range may be altered according to the exercise experience of the user or the like.

What is claimed is:

1. A biological information detector comprising:
a biological information detector configured to detect biological information of a user;
a storage configured to store the biological information;
a timepiece configured to indicate a current date-time; and
an input unit configured to receive an input operation, wherein
when a predetermined input operation is performed on the input unit, the time indicated by the timepiece is stored as an occurrence time of a hypoglycemia symptom.

2. The biological information detector according to claim 1, wherein
the predetermined input operation is a combination of a first operation and a second operation that are mutually different.

3. The biological information detector according to claim 2, wherein
the input unit comprises a tap operation detection unit configured to detect a tap operation of the user and an operation unit comprising a button exposed to an outside,
the first operation is the tap operation, and
the second operation is an input operation on the button.

4. The biological information detector according to claim 2, further comprising:
a display configured to display a setting screen allowing a transition to an occurrence timing recordation screen configured to record the occurrence time of the hypoglycemia symptom when the first operation is performed, wherein
the biological information detector is configured to store the time indicated by the timepiece as the occurrence time of the hypoglycemia symptom when the second operation is performed on the displayed setting screen.

5. The biological information detector according to claim 3, further comprising:
a display configured to display a setting screen allowing a transition to an occurrence timing recordation screen configured to record the occurrence time of the hypoglycemia symptom when the first operation is performed, wherein
the biological information detector is configured to store the time indicated by the timepiece as the occurrence time of the hypoglycemia symptom when the second operation is performed on the displayed setting screen.

6. The biological information detector according to claim 4, wherein
when the first operation is performed, the display is configured to display a setting screen configured to display a plurality of items, one of the plurality of items being configured for the transition to the occurrence timing recordation screen and being displayed preferentially to the rest of the plurality of items.

7. The biological information detector according to claim 1, further comprising:
a body movement information detection unit configured to detect body movement information of the user; and
a storage configured to store the biological information and the body movement information.

8. A biological information measurement system comprising:
the biological information detector according to claim 1; and
an information processor configured to communicate with the biological information detector, wherein
the biological information detector comprises a time information sending unit configured to send time information indicating a time stored in the storage as the occurrence time of the hypoglycemia symptom,
the information processor comprises:
an information acquisition unit configured to acquire information sent from the biological information detector;
a processor-side display configured to display an input screen, on which a time of at least one of a meal, an exercise and medicine-dose of the user is capable of being inputted; and
a time storage configured to store the time indicated by the time information acquired by the information acquisition unit and the inputted time.

9. The biological information measurement system according to claim 8, further comprising:
a timing display screen generation unit configured to generate a timing display screen comprising a time table showing hours in a predetermined time period arranged in a chronological order, a hypoglycemia occurrence marker disposed in the time table correspondingly to the time indicated by the time information, and an active time marker disposed in the time table correspondingly to the time, wherein
the processor-side display is configured to display the timing display screen.

10. The biological information measurement system according to claim 8, wherein
the time information comprises a date to which the occurrence time of the hypoglycemia symptom belongs,
the information processor comprises a calendar display screen generation unit configured to generate a calendar display screen comprising a calendar showing date in a predetermined time period arranged in a chronological order and a hypoglycemia occurrence marker disposed in the calendar correspondingly at least to the date comprised in the time information, and
the processor-side display is configured to display the calendar display screen.

11. A timing storing method performed using a biological information detector configured to measure biological information, the method comprising:
storing a current time as an occurrence time of a hypoglycemia symptom when a predetermined input operation is performed.

12. A biological information detector comprising:
a biological information detection unit configured to detect biological information of a user;
a body movement information detection unit configured to detect the body movement information and operation information of the user;
a timepiece configured to indicate date and time; and
a controller configured to acquire the biological information and the body movement information, wherein
the controller is configured to judge a condition of the user based on the biological information and the body movement information and, when it is detected that a predetermined operation is performed based on the operation information from the body movement information detection unit, judge the time indicated by the timepiece as the occurrence time of the hypoglycemia symptom.

13. The biological information detector according to claim 12, wherein
the status of the user comprises at least one of an attachment condition of the biological information detector, a type of an exercise performed by the user and an intensity of the exercise performed by the user.

14. The biological information detector according to claim 12, further comprising:
a storage configured to store the biological information, the body movement information and the occurrence time of the hypoglycemia symptom.

15. The biological information detector according to claim 12, further comprising:
a communication unit configured to send the biological information, the body movement information and the occurrence time of the hypoglycemia symptom to the information processor.

16. The biological information detector according to claim 12, wherein
the predetermined operation is a tap operation.

17. The biological information detector according to claim 16, wherein
the body movement information detection unit is an acceleration sensor, and
the controller detects the tap operation based on an output signal from the acceleration sensor.

18. A biological information presentation system comprising:
a biological information detector; and
an information processing terminal, wherein
the biological information detection unit comprises:
a biological information detection unit configured to detect biological information of a user;
a body movement information detection unit configured to detect body movement information of the user;
a timepiece configured to indicate date and time;
a control unit configured to acquire the biological information and the body movement information of the user, to judge a type and a time of an exercise performed by the user based on the biological information and the body movement information and to judge the time indicated by the timepiece as a time the occurrence time of the hypoglycemia symptom when it is detected that a predetermined operation is performed based on a signal from the body movement information detection unit; and
a communication unit configured to send exercise information comprising the type and time of the exercise performed by the user and hypoglycemia occurrence information comprising the occurrence time of the hypoglycemia symptom, and wherein
the information processing terminal comprises:
a receiver configured to receive the exercise information and the hypoglycemia occurrence information from the biological information detector; and
a display control unit configured to display an exercise time for each of a plurality of the exercise types on a first display area based on the exercise information, to display an action performed by the user comprising at least one of a medicine-dose, a meal and a sleep in a second display area in a chronological order, and to display a marker in the second display area at a position corresponding to the occurrence time of the hypoglycemia symptom when the hypoglycemia occurrence information is received from the biological information detector.

19. The biological information presentation system according to claim 18, wherein
the exercise information comprises the type of the exercise performed by the user and the exercise time for each of the plurality of exercise types.

20. The biological information presentation system according to claim 19, wherein
the display control unit displays the exercise time for each of the plurality of the exercise types in a graph in the first display area.
